(19) United States
(12) Patent Application Publication
(10) Pub. No.: US 2025/0134530 A1
(43) Pub. Date: May 1, 2025

(54) DELIVERY DEVICE FOR OCCLUSIVE IMPLANTS

(71) Applicant: Boston Scientific Scimed, Inc., Maple Grove, MN (US)

(72) Inventors: David John Onushko, Maple Grove, MN (US); Christopher J Clark, St. Michael, MN (US); Mark Christian McPhail, Maple Grove, MN (US); Joseph Edward Adriaens, Elk River, MN (US)

(73) Assignee: Boston Scientific Scimed, Inc., Maple Grove, MN (US)

(21) Appl. No.: 18/930,183

(22) Filed: Oct. 29, 2024

Related U.S. Application Data
(60) Provisional application No. 63/594,149, filed on Oct. 30, 2023.

(51) Int. Cl. A61B 17/12

(52) U.S. Cl. CPC A61B 17/12172; A61B 17/12122; A61B 2017/1205

(57) ABSTRACT

Delivery devices for an occlusive implant and method for making and using delivery devices are disclosed. An example delivery device may include a delivery sheath having a proximal region, a distal tip region, and a lumen formed therein. A core wire may be slidably disposed in the lumen. The core wire may include a central shaft, a first region having a first coil assembly disposed along the central shaft, a second region having a second coil assembly disposed along the central shaft, and a distal implant securing region. An occlusive implant may be coupled to the distal implant securing region.

### CROSS-REFERENCE TO RELATED APPLICATIONS

The present application claims the benefit of U.S. Provisional Patent Application Ser. No. 63/594,149, filed Oct. 30, 2023, the disclosure of which is incorporated herein by reference.

### TECHNICAL FIELD

The present disclosure pertains to medical devices, and methods for manufacturing medical devices. More particularly, the present disclosure pertains to delivery devices for occlusive implants.

### BACKGROUND

A wide variety of medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A delivery device for an occlusive implant is disclosed. The delivery device comprises: a delivery sheath having a proximal region, a distal tip region, and a lumen formed therein; a core wire slidably disposed in the lumen; wherein the core wire includes a central shaft, a first region having a first coil assembly disposed along the central shaft, a second region having a second coil assembly disposed along the central shaft, and a distal implant securing region; and an occlusive implant coupled to the distal implant securing region.

Alternatively or additionally to any of the embodiments above, the central shaft includes stainless steel.

Alternatively or additionally to any of the embodiments above, the central shaft includes a nickel-titanium alloy.

Alternatively or additionally to any of the embodiments above, the central shaft includes a stainless steel member and a nickel-titanium alloy member secured to the stainless steel member.

Alternatively or additionally to any of the embodiments above, the first region includes an outer jacket disposed over the first coil assembly.

Alternatively or additionally to any of the embodiments above, the first region includes a braid coupled to the first coil assembly.

Alternatively or additionally to any of the embodiments above, the first coil assembly includes a single coil.

Alternatively or additionally to any of the embodiments above, the first coil assembly includes two or more coils.

Alternatively or additionally to any of the embodiments above, the second coil assembly includes two or more coils.

Alternatively or additionally to any of the embodiments above, further comprising one or more bead members disposed adjacent to the first region.

Alternatively or additionally to any of the embodiments above, further comprising one or more bead members disposed between the first region and the second region.

Alternatively or additionally to any of the embodiments above, the first region has a first outer diameter and wherein the second region has a second outer diameter different from the first outer diameter.

A delivery device for an occlusive implant is disclosed. The delivery device comprises: a delivery sheath having a proximal region, a distal tip region, and a lumen formed therein; a core wire slidably disposed in the lumen; wherein the core wire includes a central shaft, a first region having a coil assembly disposed along the central shaft, a second region having spacer assembly disposed along the central shaft, and a distal implant securing region; and an occlusive implant coupled to the distal implant securing region.

Alternatively or additionally to any of the embodiments above, the spacer assembly includes one or more bead members.

Alternatively or additionally to any of the embodiments above, the spacer assembly includes a stent-like structure.

Alternatively or additionally to any of the embodiments above, the spacer assembly includes a spiral tube disposed about the central shaft.

Alternatively or additionally to any of the embodiments above, the central shaft includes stainless steel.

Alternatively or additionally to any of the embodiments above, the central shaft includes a stainless steel member and a nickel-titanium alloy member secured to the stainless steel member.

Alternatively or additionally to any of the embodiments above, the delivery sheath has an inner diameter and wherein spacer member has an outer diameter that closely approximates the inner diameter.

A method for implanting an occlusive implant into a left atrial appendage is disclosed. The method comprises: advancing a delivery device to a position adjacent to the left atrial appendage, the delivery device comprising: a delivery sheath having a proximal region, a distal tip region, and a lumen formed therein, a core wire slidably disposed in the lumen, wherein the core wire includes a central shaft, a first region having a first coil assembly disposed along the central shaft, a second region having a second coil assembly disposed along the central shaft, and a distal implant securing region, and an occlusive implant coupled to the distal implant securing region; and advancing the core wire such that the occlusive implant advances out from the distal tip region of the delivery sheath.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of an example delivery system.

FIG. 2 is a side view of an example delivery system.

FIG. 3 is a side view of a portion of an example delivery system.

FIG. 4 is a side view of a portion of an example delivery system.

FIG. 5 is a cross-sectional view taken through line 5-5 in FIG. 4.

FIG. 6 is a cross-sectional view of a portion of an example delivery system.

FIG. 7 is a cross-sectional view of a portion of an example delivery system.

FIG. 8 is a side view of a portion of an example delivery system.

FIG. 9 is a side view of a portion of an example delivery system.

FIG. 10 is a side view of a portion of an example delivery system.

FIG. 11 is a side view of a portion of an example delivery system.

FIG. 12 is a side view of a portion of an example delivery system.

FIG. 13 is a side view of a portion of an example delivery system.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

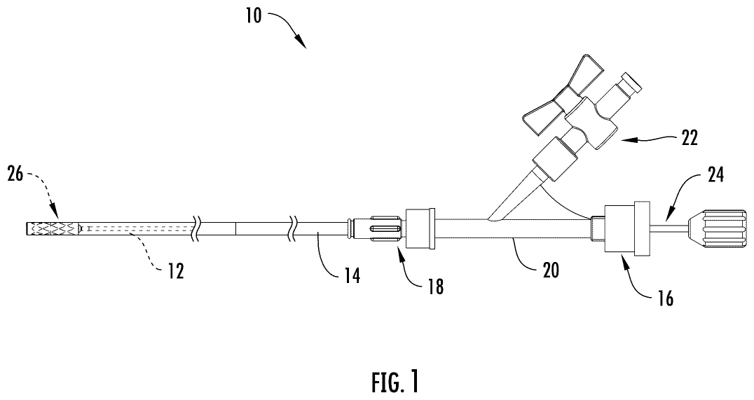

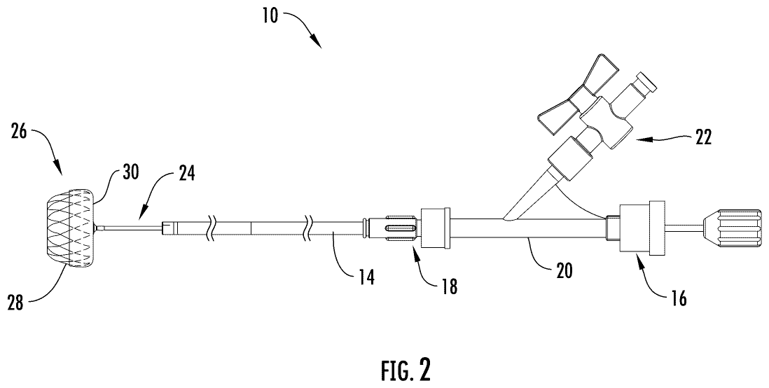

FIGS. 1-2 schematically illustrate selected components and/or arrangements of an occlusive implant system. It should be noted that in any given figure, some features of the occlusive implant system may not be shown, or may be shown schematically, for simplicity. Additional details regarding some of the components of the occlusive implant system may be illustrated in other figures in greater detail. The occlusive implant system may be used to deliver and/or deploy a variety of medical implants (e.g., a cardiovascular implant, an occlusive implant, etc.) to one or more locations within the anatomy, including but not limited to, in some embodiments, the heart and/or the left atrial appendage. In the interest of clarity, the following discussion refers to an occlusive implant, but other medical implants may be used and/or considered with the occlusive implant system.

The occlusive implant system may include a delivery system 10 including a delivery sheath 14 having a delivery lumen 12 extending proximally from a distal end of the delivery sheath 14. In one example, the delivery lumen 12 extends from a proximal opening to a distal opening of the delivery sheath 14. The delivery system 10 may include a proximal hub 16. In some embodiments, the delivery system may include a mid-hub 18. In some embodiments, the delivery system 10 may include a mid-shaft 20 extending from the proximal hub 16 to the mid-hub 18. In some embodiments, the delivery sheath 14 may extend distally from the mid-hub 18. Other configurations are also contemplated. In some embodiments, the delivery system 10 may include a side port 22. In some embodiments, the side port 22 may be in communication with the mid-shaft 20. Other configurations are also contemplated. In some embodiments, the delivery system 10 and/or the delivery lumen 12 may include a proximal segment (not shown) extending within and/or through the mid-hub 18, the mid-shaft 20, and the proximal hub 16. In some embodiments, the proximal segment may be in fluid communication with and/or may be an extension of the delivery lumen 12 of the delivery sheath 14. In some embodiments, the side port 22 may be in fluid communication with the proximal segment and/or the delivery lumen 12.

The occlusive implant system and/or the delivery system 10 may include a core member or core wire 24 slidably and/or rotatably disposed within the delivery lumen 12 (and the proximal segment, where present). The occlusive implant system may include an occlusive implant 26, which may be configured for implantation within a left atrial appendage, releasably engaged with and/or releasably attached to a distal end of the core wire 24. In at least some embodiments, the occlusive implant 26 may be a left atrial appendage closure device. In some embodiments, a proximal end of the core wire 24 may extend proximally of a proximal end of the delivery sheath 14 and/or the proximal opening of the delivery lumen 12 for manual manipulation by a clinician or practitioner. In at least some embodiments, the delivery sheath 14 may comprise and/or may be formed from a polymeric material. In some embodiments, the delivery sheath 14 may comprise and/or may be formed from a plurality of polymeric materials. In some embodiments, the delivery sheath may comprise and/or may be formed from a combination of metallic and polymeric materials. In some embodiments, the delivery sheath 14 may include a reinforcing element, such as a mesh, a coil, a braid, etc., formed therein, embedded therein, attached thereto, etc. along at least a portion of a length of the delivery sheath 14. Other configurations are also contemplated. Some suitable, but non-limiting, examples of materials for the occlusive implant system, the core wire 24, and/or the delivery sheath 14, etc., including but not limited to metallic materials, polymeric materials, etc., are discussed below.

The occlusive implant 26 may include an expandable framework 28 (e.g., FIG. 2) configured to shift between a delivery configuration (e.g., FIG. 1), such as when the occlusive implant 26 is disposed within the delivery lumen 12 proximate the distal opening and/or within a distal portion of the delivery lumen 12, and a deployed configuration (e.g., FIG. 2) when the occlusive implant 26 is unconstrained by the delivery sheath 14.

In some embodiments, the expandable framework 28 may comprise a plurality of interconnected struts. In some embodiments, the expandable framework 28 may be compliant or semi-compliant and may generally conform to and/or be configured to sealingly engage with the shape and/or geometry of the left atrial appendage in the deployed configuration.

In some embodiments, a proximal end of the expandable framework 28 may be configured to releasably attach, join, couple, engage, or otherwise connect to the distal end of the core wire 24 (e.g., FIG. 2). In some embodiments, the proximal end of the expandable framework 28 may include a proximal hub coupled and/or non-releasably attached thereto. In some embodiments, the proximal hub may be configured to and/or adapted to releasably couple with, join to, mate with, or otherwise engage a distal end of the core wire 24. Other means of releasably coupling and/or engaging the expandable framework 28 to the distal end of the core wire 24 are also contemplated.

In some embodiments, the occlusive implant 26 may include an occlusive element 30 (e.g., a membrane, a fabric, or a tissue element, etc.) connected to, disposed on, disposed over, disposed about, or covering at least a portion the expandable framework 28. In some embodiments, the occlusive element 30 may be connected to, disposed on, disposed over, disposed about, or cover at least a portion of an outer (or outwardly facing) surface of the expandable framework 28.

In some embodiments, the occlusive element 30 may be permeable or impermeable to blood and/or other fluids, such as water. In some embodiments, the occlusive element 30 may include a polymeric membrane, a metallic or polymeric mesh, a porous or semi-porous filter-like material, or other suitable construction. In some embodiments, the occlusive element 30 prevents thrombi (e.g., blood clots, etc.) from passing through the occlusive element 30 and out of the left atrial appendage into the blood stream. In some embodiments, the occlusive element 30 promotes endothelization after implantation, thereby effectively removing the target site (e.g., the left atrial appendage, etc.) from the patient's circulatory system. Some suitable, but non-limiting, examples of materials for the occlusive element 30 are discussed below.

In some embodiments, the expandable framework 28 and/or the plurality of interconnected struts may be integrally formed and/or cut from a unitary member. In some embodiments, the expandable framework 28 and/or the plurality of interconnected struts may be integrally formed and/or cut from a unitary tubular member and subsequently formed and/or heat set to a desired shape in the deployed configuration. In some embodiments, the expandable framework 28 and/or the plurality of interconnected struts may be integrally formed and/or cut from a unitary flat member or sheet, and then rolled or formed into a tubular structure and subsequently formed and/or heat set to the desired shape in the deployed configuration. Some exemplary means and/or methods of making and/or forming the expandable framework 28 include laser cutting, machining, punching, stamping, electro discharge machining (EDM), chemical dissolution, etc. Other means and/or methods are also contemplated.

In use, the delivery sheath 14 may be advanced and/or navigated to the left atrial appendage to deliver the occlusive implant 26 thereto. In one example, the delivery sheath 14 may be advanced and/or navigated to the left atrial appendage using and/or over a guidewire. For example, the delivery sheath 14 may be advanced to the patient's left atrium and the distal end disposed adjacent to the left atrial appendage with the occlusive implant 26 disposed therein in the delivery configuration. In some embodiments, the delivery sheath 14 may include steering capability. After the distal end of the delivery sheath 14 is disposed adjacent to and/or at the left atrial appendage, the core wire 24 may be advanced distally relative to the delivery sheath 14 to advance the occlusive implant 26 out of the delivery sheath 14, where the occlusive implant 26 may shift to the deployed configuration.

While not expressly illustrated, in some embodiments, the occlusive implant system may further comprise an access device. In some embodiments, the access device may be a bi-directional steerable catheter and/or an intravascular catheter. Examples of intravascular catheters may include, but are not limited to, balloon catheters, atherectomy catheters, device delivery catheters, drug delivery catheters, diagnostic catheters, and guide catheters.

In some embodiments, the access device may be advanced and/or navigated to the left atrial appendage. In one example, the access device may be advanced and/or navigated to the left atrial appendage using and/or over a guidewire. For example, the access device may be advanced to the patient's left atrium and a distal tip disposed adjacent to the left atrial appendage. In some embodiments, the access device may include steering capability. In some embodiments, the delivery system 10 may be inserted through the access device. In some embodiments, the length of the delivery sheath 14 may be substantially equal to the length of the access device. In some embodiments, the length of the delivery sheath 14 may be slightly longer than the access device. During use, the delivery sheath 14 may be advanced within the access device with the occlusive implant 26 disposed therein in the delivery configuration. After the distal end of the delivery sheath 14 is disposed adjacent to and/or at the distal end of the access device, the core wire 24 may be advanced distally relative to the delivery sheath 14 and/or the access device to advance the occlusive implant 26 out of the delivery sheath 14 and the access device, where the occlusive implant 26 may shift to the deployed configuration.

In some embodiments, the delivery system, the delivery sheath 14, and/or the access device may be sized in accordance with its intended use. For example, the delivery system, the delivery sheath 14, and/or the access device can have a length that is in the range of about 10 to about 150 centimeters, about 25 to about 125 centimeters, about 50 to about 100 centimeters, about 25 centimeters to about 50 centimeters, about 50 to about 75 centimeters, about 75 to about 100 centimeters, etc. Other lengths are also contemplated, including but not limited to subsets of ranges disclosed herein. It is further contemplated that the outer diameter of the delivery system, the delivery sheath 14, and/or the access device may vary based on the use or application. In some examples, the outer diameter of the delivery system, the delivery sheath 14, and/or the access device may be about 2 millimeters (mm), about 3 mm (or 9 French), about 3.5 mm, about 4 mm (or 12 French), about 4.5 mm, about 5 mm (or 15 French), about 5.33 mm, about 5.5 mm, about 5.66 mm (or 17 French), about 6 mm, about 6.5 mm, about 7 mm (or 21 French), about 8 mm, or other suitable sizes. In some embodiments, the outer diameter of the delivery system, the delivery sheath 14, and/or the access device may be a maximum of 5.66 mm (17 French) and is preferably smaller than 5.66 mm (17 French). Other configurations are also contemplated. In some embodiments, it is desirable for the outer diameter of the delivery system, the delivery sheath 14, and/or the access device to be as small as possible.

In some instances, during a procedure, it may be necessary to recapture and/or reposition the occlusive implant 26. For example, the initial placement of the occlusive implant 26 may be incorrect and/or inadequate. Accordingly, the delivery sheath 14 and/or the access device may be configured to permit recapture and/or reposition of the occlusive implant 26. Disclosed herein are delivery systems that include a delivery sheath, for example, with proximal region, a garage region, and a distal tip region. These and/or other structures may help to make the delivery sheath more amenable to recapture and/or repositioning of the occlusive implant. Some additional details regarding these and other structures are disclosed herein.

It can be appreciated that delivery of the occlusive implant may include pushing the occlusive implant through the delivery sheath with the core wire. In order to efficiently deliver the occlusive implant, it may be desirable to manufacture the core wire to include a desirable level of push support (e.g., pushability). In addition, because the inner diameter of the delivery sheath may be larger than the outer diameter of some core wires, the core wire could shift or bend within the delivery sheath. This could reduce the control and/or precision of delivering the occlusive implant and/or increase jumping of the occlusive implant. The systems and core wires disclosed herein are designed to help increase deliverability of occlusive implants. Some additional details of the features and benefits such systems (and/or core wires) are disclosed herein.

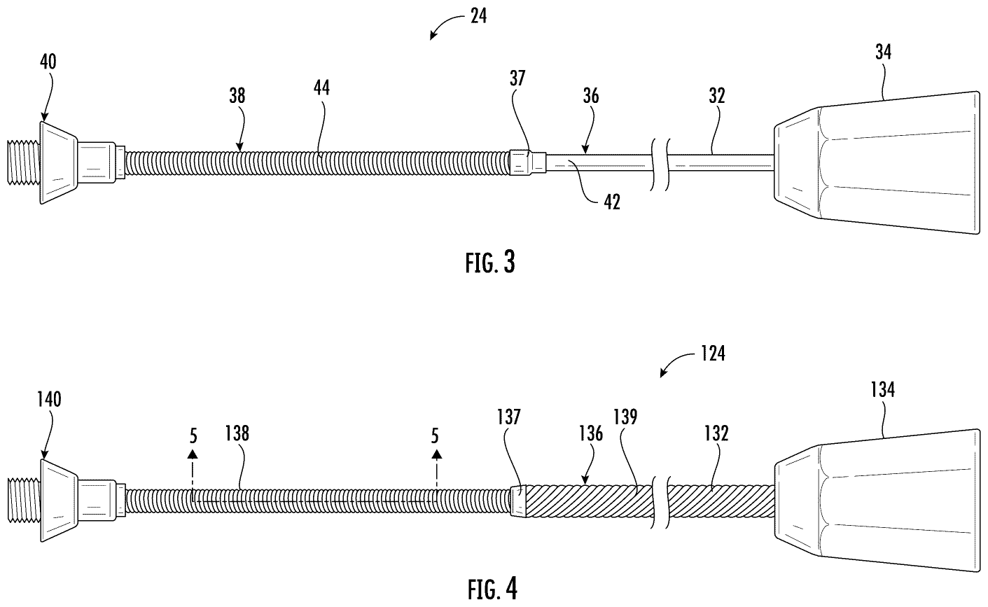

FIG. 3 illustrates the core wire 24. In general, the core wire 24 is designed to have a desirable level of push support (e.g., pushability) so as to enhance the ability of the delivery system 10 to efficiently and precisely deliver the occlusive implant 26. Here it can be seen that that the core wire 24 may include a proximal shaft region 36 having a proximal end region 32. A handle member 34 may be coupled to the proximal end region 32. The proximal shaft region 36 may include a central shaft 42 formed from a relatively stiff material such as stainless steel, a nickel-titanium alloy (e.g., which may include a superelastic or linear elastic nickel-titanium alloy), and/or the like. A relatively stiff central shaft 42 may provide a desirable level of push support (e.g., pushability) to the core wire 24. Other materials are contemplated including those disclosed herein. In some instances, the central shaft 42 may include a first or proximal section and a second or distal section coupled to the first section. For example, the central shaft 42 may be include a proximal stainless steel section that is coupled to (e.g., thermally bonded, mechanically bonded, adhesively bonded, welded, swaged, combinations therefor, and/or the like) a distal nickel-titanium alloy section.

The core wire 24 may also include a distal shaft region 38. As shown in FIG. 3, the distal shaft region 38 may include a coil or coil assembly 44. The coil or coil assembly 44 may include a single coil. Alternatively, the coil assembly 44 may include two or more coils (e.g., opposed coils). In at least some instances, the coil 44 may be disposed about the central shaft 42. In other words, the central shaft 42 may extend along the distal shaft region 38 and the coil 44 may be disposed about the central shaft 42 along the distal shaft region 38. The coil 44 may provide an increased level of push support along the distal shaft region 38 while still being highly flexible. In addition, the coil 44 may increase the outer diameter of the core wire 24 along the distal shaft region 38. For example, the coil 44 may define an outer diameter that approximates the inner diameter of the delivery sheath 14. This may help to reduce the likelihood that the core wire 24 can shift, jump, or otherwise foreshorten within the delivery sheath 14. While not expressly shown, an outer coating or sleeve may be disposed along the coil 44. The sleeve or coating may include a lubricious coating/sleeve. In some instances, a proximal end region of the coil 44 may be coupled to the central shaft 42 at and/or be a transition or transition member 37. The transition member 37 may take the form of a sleeve, weld, adhesive bond, combinations thereof, and/or the like. A distal connector 40 may be coupled to the distal shaft region 38. In at least some instances, the distal connector 40 may be configured to secure the core wire 24 to the occlusive implant 26.

FIGS. 4-5 illustrate another example core wire 124 that may be similar in form and function to other core wires disclosed herein. The core wire 124 may be used with the delivery system 10 and/or other similar systems including those disclosed herein. In general, the core wire 124 is designed to have a desirable level of push support (e.g., pushability) so as to enhance the ability of the delivery system 10 to efficiently and precisely deliver the occlusive implant 26. Here it can be seen that that the core wire 124 may include a proximal shaft region 136 having a proximal end region 132. A handle member 134 may be coupled to the proximal end region 132. The core wire 124 may include a central shaft 142 (see, for example, FIG. 5) formed from a relatively stiff material such as stainless steel, a nickel-titanium alloy (e.g., which may include a superelastic or linear elastic nickel-titanium alloy), and/or the like. A relatively stiff central shaft 142 may provide a desirable level of push support (e.g., pushability) to the core wire 124. Other materials are contemplated including those disclosed herein. In some instances, the central shaft 142 may include a first or proximal section and a second or distal section coupled to the first section. For example, the central shaft 142 may be include a proximal stainless steel section that is coupled to (e.g., thermally bonded, mechanically bonded, adhesively bonded, welded, swaged, combinations therefor, and/or the like) a distal nickel-titanium alloy section.

The core wire 124 may also include distal shaft region 138. The distal shaft region 138 may include a coil 144 (e.g., a coil or coil assembly 144 that may include a single coil or two or more coils). In at least some instances, the coil 144 may be disposed about the central shaft 142 (see, for example, FIG. 5). In other words, the central shaft 142 may extend along the distal shaft region 138 and the coil 144 may be disposed about the central shaft 142 along the distal shaft region 138. The coil 144 may provide an increased level of push support along the distal shaft region 138 while still being highly flexible. In addition, the coil 144 may increase the outer diameter of the core wire 124 along the distal shaft region 38. For example, the coil 144 may define an outer diameter that approximates the inner diameter of the delivery sheath 14. This may help to reduce the likelihood that the core wire 124 can shift, jump, or otherwise foreshorten within the delivery sheath 114. While not expressly shown, an outer coating or sleeve may be disposed along the coil 144. The sleeve or coating may include a lubricious coating/sleeve. In some instances, a proximal end region of the coil 144 may be coupled to the central shaft 142 at and/or be a transition or transition member 137. The transition member 137 may take the form of a sleeve, weld, adhesive bond, combinations thereof, and/or the like. the proximal shaft region 136 may also include a coil or coil assembly 139. In some instances, the coil assembly 139 may include one or more coils (e.g., such as a pair of opposed coils). While not expressly shown, an outer coating or sleeve may be disposed along the coil assembly 139. A distal connector 140 may be coupled to the distal shaft region 138. In at least some instances, the distal connector 140 may be configured to secure the core wire 124 to the occlusive implant 26.

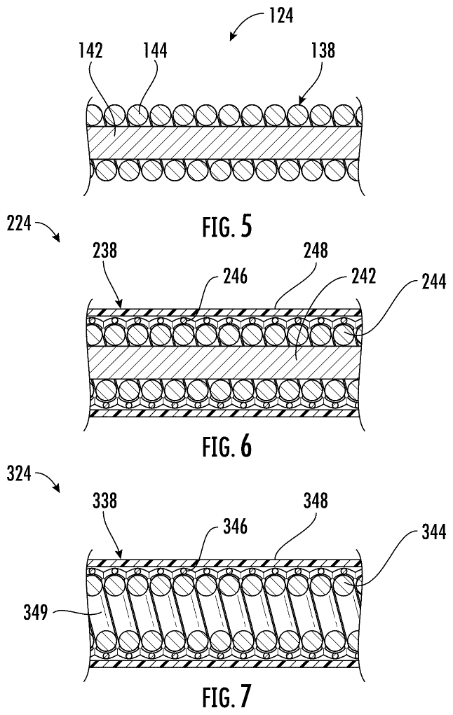

FIG. 6 illustrates a portion of another example core wire 224 that may be similar in form and function to other core wires disclosed herein. The core wire 224 may be used with the delivery system 10 and/or other similar systems including those disclosed herein. In this example, a portion of the distal shaft region 238 is shown. The distal shaft region 238 may include a central shaft 242, a coil 244 disposed about the central shaft 242, and a braid member 246 disposed over the coil 244. A sleeve 248 may be disposed over the coil 244 and the braid member 246. An assembly such as what is shown in FIG. 6 can be used for distal and/or proximal shaft sections, as appropriate, of suitable core wires disclosed herein.

FIG. 7 illustrates a portion of another example core wire 324 that may be similar in form and function to other core wires disclosed herein. The core wire 324 may be used with the delivery system 10 and/or other similar systems including those disclosed herein. In this example, a portion of the distal shaft region 338 is shown. The distal shaft region 338 may include a coil 344 and a braid member 346 disposed over the coil 344. A lumen 349 may be defined within the coil 344. A sleeve 348 may be disposed over the coil 344 and the braid member 346. An assembly such as what is shown in FIG. 7 can be used for distal and/or proximal shaft sections of suitable core wires disclosed herein.

FIG. 8 illustrates a portion of another example core wire 424 that may be similar in form and function to other core wires disclosed herein. The core wire 424 may be used with the delivery system 10 and/or other similar systems including those disclosed herein. In this example, a portion of the distal shaft region 438 is shown. The distal shaft region 438 may include a central shaft 442 and a spiral/helical tube 446 disposed along the central shaft 442. The spiral/helical tube 446 may increase the outer diameter of the core wire 424 along the distal shaft region 438. For example, the spiral/helical tube 446 may define an outer diameter that approximates the inner diameter of the delivery sheath 14. This may help to reduce the likelihood that the core wire 424 can shift, jump, or otherwise foreshorten within the delivery sheath 14. While not expressly shown, an outer coating or sleeve may be disposed along the spiral/helical tube 446.

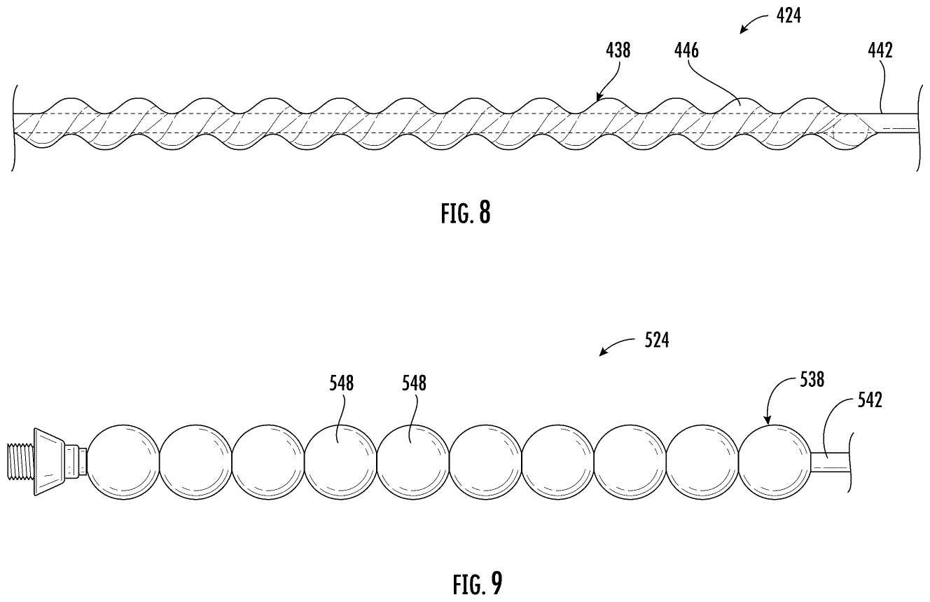

FIG. 9 illustrates a portion of another example core wire 524 that may be similar in form and function to other core wires disclosed herein. The core wire 524 may be used with the delivery system 10 and/or other similar systems including those disclosed herein. In this example, a portion of the distal shaft region 538 is shown. The distal shaft region 438 may include a central shaft 542 and one or more bead members 548 disposed along the central shaft 542. The bead members 548 may increase the outer diameter of the core wire 524 along the distal shaft region 538. For example, the bead members 548 may define an outer diameter that approximates the inner diameter of the delivery sheath 14. This may help to reduce the likelihood that the core wire 524 can shift, jump, or otherwise foreshorten within the delivery sheath 14. While not expressly shown, an outer coating or sleeve may be disposed along the spiral/helical tube 546.

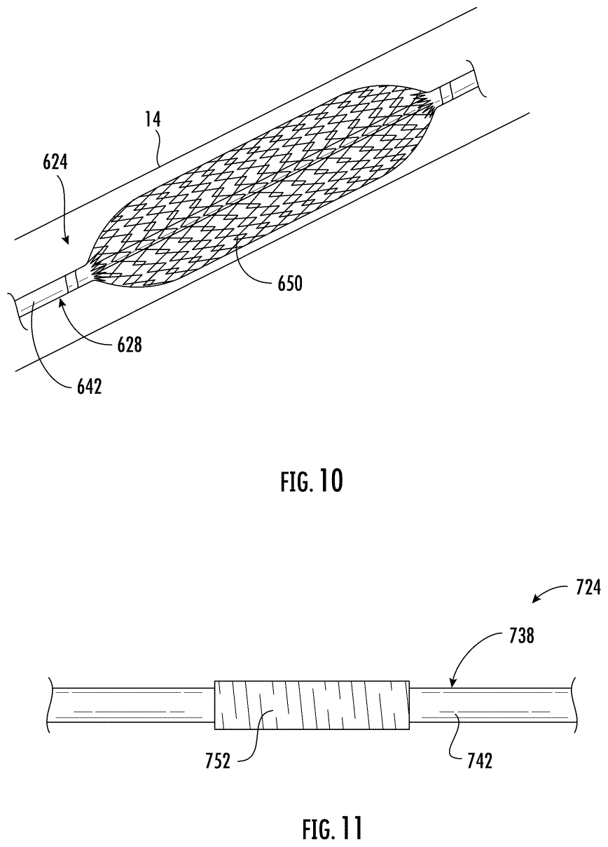

FIG. 10 illustrates a portion of another example core wire 624 that may be similar in form and function to other core wires disclosed herein. The core wire 624 may be used with the delivery system 10 and/or other similar systems including those disclosed herein. In this example, a portion of the distal shaft region 638 is shown. The distal shaft region 638 may include a central shaft 642 and a stent-like structure 650 disposed along the central shaft 642. The stent-like structure 650 may increase the outer diameter of the core wire 624 along the distal shaft region 638. For example, the stent-like structure 650 may define an outer diameter that approximates the inner diameter of the delivery sheath 14. This may help to reduce the likelihood that the core wire 624 can shift, jump, or otherwise foreshorten within the delivery sheath 14. While not expressly shown, an outer coating or sleeve may be disposed along the stent-like structure 650.

FIG. 11 illustrates a portion of another example core wire 724 that may be similar in form and function to other core wires disclosed herein. The core wire 724 may be used with the delivery system 10 and/or other similar systems including those disclosed herein. In this example, a portion of the distal shaft region 738 is shown. The distal shaft region 738 may include a central shaft 742 and a slotted tubular member 752 disposed along the central shaft 742. The slotted tubular member 752 may increase the outer diameter of the core wire 724 along the distal shaft region 738. For example, the slotted tubular member 752 may define an outer diameter that approximates the inner diameter of the delivery sheath 14. This may help to reduce the likelihood that the core wire 724 can shift, jump, or otherwise foreshorten within the delivery sheath 14. While not expressly shown, an outer coating or sleeve may be disposed along the slotted tubular member 752.

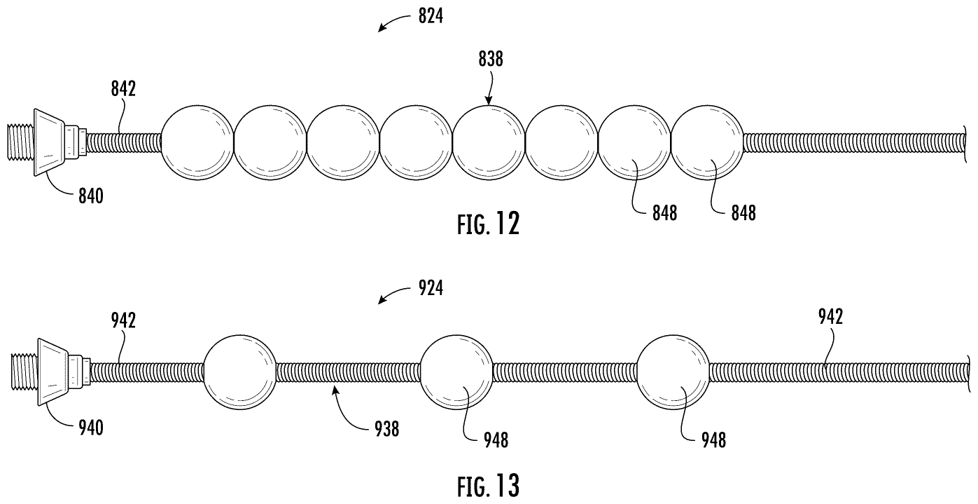

FIG. 12 illustrates a portion of another example core wire 824 that may be similar in form and function to other core wires disclosed herein. The core wire 724 may be used with the delivery system 10 and/or other similar systems including those disclosed herein. In this example, a distal shaft region 838 and a distal connector 840 are shown. The distal shaft region 838 may include a central shaft 842. One or more bead members 848 may be disposed along the central shaft 842 (e.g., along the coil). In this example, at least some of the bead members 828 may be disposed adjacent to (e.g., in contact with) one another. The bead members 828 may increase the outer diameter of the core wire 824 along the distal shaft region 838. For example, the bead members 828 may define an outer diameter that approximates the inner diameter of the delivery sheath 14. This may help to reduce the likelihood that the core wire 824 can shift, jump, or otherwise foreshorten within the delivery sheath 14. While not expressly shown, an outer coating or sleeve may be disposed along the bead members 828.

FIG. 13 illustrates a portion of another example core wire 924 that may be similar in form and function to other core wires disclosed herein. The core wire 924 may be used with the delivery system 10 and/or other similar systems including those disclosed herein. In this example, a distal shaft region 938 and a distal connector 940 are shown. The distal shaft region 938 may include a central shaft 942. One or more bead members 948 may be disposed along the central shaft 942 (e.g., along the coil). In this example, at least some of the bead members 928 may be spaced apart from one another. The bead members 928 may increase the outer diameter of the core wire 924 along the distal shaft region 938. For example, the bead members 828 may define an outer diameter that approximates the inner diameter of the delivery sheath 14. This may help to reduce the likelihood that the core wire 924 can shift, jump, or otherwise foreshorten within the delivery sheath 14. While not expressly shown, an outer coating or sleeve may be disposed along the bead members 928.

The materials that can be used for the various components of the delivery system 10 (and/or other delivery systems disclosed herein) may include those commonly associated with medical devices. For simplicity purposes, the following discussion makes reference to the delivery system 10. However, this is not intended to limit the devices and methods described herein, as the discussion may be applied to other delivery systems disclosed herein.

The delivery system 10 and/or other components of the delivery system 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly (alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the delivery system 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the delivery system 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the delivery system 10 to achieve the same result.

In some embodiments, a degree of Magnetic Resonance Imaging (MRI) compatibility is imparted into the delivery system 10. For example, the delivery system 10, or portions thereof, may be made of a material that does not substantially distort the image and create substantial artifacts (e.g., gaps in the image). Certain ferromagnetic materials, for example, may not be suitable because they may create artifacts in an MRI image. The delivery system 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A delivery device for an occlusive implant, the delivery device comprising:
a delivery sheath having a proximal region, a distal tip region, and a lumen formed therein;
a core wire slidably disposed in the lumen;
wherein the core wire includes a central shaft, a first region having a first coil assembly disposed along the central shaft, a second region having a second coil assembly disposed along the central shaft, and a distal implant securing region; and
an occlusive implant coupled to the distal implant securing region.

2. The delivery device of claim 1, wherein the central shaft includes stainless steel.

3. The delivery device of claim 1, wherein the central shaft includes a nickel-titanium alloy.

4. The delivery device of claim 1, wherein the central shaft includes a stainless steel member and a nickel-titanium alloy member secured to the stainless steel member.

5. The delivery device of claim 1, wherein the first region includes an outer jacket disposed over the first coil assembly.

6. The delivery device of claim 1, wherein the first region includes a braid coupled to the first coil assembly.

7. The delivery device of claim 1, wherein the first coil assembly includes a single coil.

8. The delivery device of claim 1, wherein the first coil assembly includes two or more coils.

9. The delivery device of claim 1, wherein the second coil assembly includes two or more coils.

10. The delivery device of claim 1, further comprising one or more bead members disposed adjacent to the first region.

11. The delivery device of claim 1, further comprising one or more bead members disposed between the first region and the second region.

12. The delivery device of claim 1, wherein the first region has a first outer diameter and wherein the second region has a second outer diameter different from the first outer diameter.

13. A delivery device for an occlusive implant, the delivery device comprising:
a delivery sheath having a proximal region, a distal tip region, and a lumen formed therein;
a core wire slidably disposed in the lumen;
wherein the core wire includes a central shaft, a first region having a coil assembly disposed along the central shaft, a second region having spacer assembly disposed along the central shaft, and a distal implant securing region; and
an occlusive implant coupled to the distal implant securing region.

14. The delivery device of claim 13, wherein the spacer assembly includes one or more bead members.

15. The delivery device of claim 13, wherein the spacer assembly includes a stent-like structure.

16. The delivery device of claim 13, wherein the spacer assembly includes a spiral tube disposed about the central shaft.

17. The delivery device of claim 13, wherein the central shaft includes stainless steel.

18. The delivery device of claim 13, wherein the central shaft includes a stainless steel member and a nickel-titanium alloy member secured to the stainless steel member.

19. The delivery device of claim 13, wherein the delivery sheath has an inner diameter and wherein spacer member has an outer diameter that closely approximates the inner diameter.

20. A method for implanting an occlusive implant into a left atrial appendage, the method comprising:
advancing a delivery device to a position adjacent to the left atrial appendage, the delivery device comprising:
a delivery sheath having a proximal region, a distal tip region, and a lumen formed therein,
a core wire slidably disposed in the lumen,
wherein the core wire includes a central shaft, a first region having a first coil assembly disposed along the central shaft, a second region having a second coil assembly disposed along the central shaft, and a distal implant securing region, and
an occlusive implant coupled to the distal implant securing region; and
advancing the core wire such that the occlusive implant advances out from the distal tip region of the delivery sheath.
